(19) United States
(12) Patent Application Publication
(10) Pub. No.: US 2018/0098876 A1
(43) Pub. Date: Apr. 12, 2018

(54) OSTOMY APPLIANCE WITH FASTENING PADS AND RELATED METHODS

(71) Applicant: BETTER OSTOMY, LLC, Winter Park, FL (US)

(72) Inventor: Charles Anthony WRIGHT, Winter Park, FL (US)

(21) Appl. No.: 15/578,851

(22) PCT Filed: May 25, 2016
(86) PCT No.: PCT/US2016/034094

§ 371 Date: Dec. 1, 2017

Related U.S. Application Data
(60) Provisional application No. 62/171,647, filed on Jun. 5, 2015.

(51) Int. Cl. A61F 5/448

(52) U.S. Cl. CPC A61F 5/448; A61F 5/449; A61F 2005/4486

(57) ABSTRACT

An ostomy appliance may include a wafer base plate to be coupled to a stoma of a patient and having a base layer with an opening positioned inwardly adjacent edges of the base layer, and first fastening pads carried by the base layer. The ostomy appliance may include an ostomy bag assembly to be coupled to the wafer base plate and having a bag with an opening to receive waste from the stoma of the patient, the opening being positioned inwardly from adjacent edges of the bag, and second fastening pads carried by the bag and configured to couple to the first fastening pads so that corresponding portions of the bag are retained against adjacent portions of the wafer base plate as the bag fills with the waste.

### RELATED APPLICATION

This application is based upon prior filed copending Application No. 62/171,647 filed Jun. 5, 2015, the entire subject matter of which is incorporated herein by reference in its entirety.

### TECHNICAL FIELD

The present disclosure relates to the field of ostomy appliances, and, more particularly, to an ostomy bag and related methods.

### BACKGROUND

An ostomy pouching system is a prosthetic medical device that provides a means for the collection of waste from a surgically diverted biological system (i.e. colon, ileum, and bladder), which terminates at a stoma. Pouching systems are most commonly associated with colostomies, ileostomies, and urostomies.

Pouching systems, known as appliances, comprise either a one-piece system involving a plastic bag type collection pouch or a two-piece system involving a mounting plate, commonly called a flange, wafer or a base plate, and a reusable or one time useable and disposable collection plastic bag type collection pouch that is attached mechanically or with an adhesive mounting plate to create an airtight seal. The selection of systems varies greatly between individuals and is most often based on personal preference and lifestyle.

Numerous circumstances can occur that can affect the fit and appearance of the appliance system. Factors that can contribute to a changed fitting profile are varied, such as: the top of the pouch will fold over or pucker as waste collects in the pouch increasing pouch bulkiness, the actual physical stoma location on the body, the area around the stoma may become distended due to hernia, unvented gas accumulation within the pouch and the stoma itself may change in size and shape. Whatever the event, the ostomate is very aware of changes that can affect the appearance of his/her appliance.

Every ostomate is continuously concerned with several important issues related to his/her ostomy, which because of the personally sensitive nature is not generally expressed to others. Two key issues pertain to personal appearance rather than being medically-related. These issues are: “What changes in my clothing will I have to make to cover my appliance?” and “Is my appliance apparent to others?”

### SUMMARY

In view of the foregoing background, it is therefore an object of the present disclosure to provide an ostomy appliance that is robust and readily used while easily concealed.

This and other objects, features, and advantages in accordance with the present disclosure are provided by an ostomy appliance that may comprise a wafer base plate to be coupled to a stoma of a patient and comprising a base layer having an opening therein positioned inwardly adjacent edges of the base layer, a first ring sealing feature carried by the base layer and surrounding the opening, and a plurality of first fastening pads carried by the base layer surrounding the first sealing feature. The ostomy appliance may include an ostomy bag assembly to be coupled to the wafer base plate and comprising a bag having an opening to receive waste from the stoma of the patient, the opening being positioned inwardly from adjacent edges of the bag, a second ring sealing feature to be coupled to the first ring sealing feature and surrounding the opening of the bag, and a plurality of second fastening pads carried by the bag surrounding the second ring sealing feature. The plurality of second fastening pads may be configured to couple to the plurality of first fastening pads so that corresponding portions of the bag are retained against adjacent portions of the wafer base plate as the bag fills with the waste. Advantageously, the ostomy appliance will not sag and pucker visibly as the bag fills.

In some embodiments, the plurality of first fastening pads may comprise a plurality of first Velcro pads, and the plurality of second fastening pads may comprise a plurality of second Velcro pads. In other embodiments, the plurality of first fastening pads may include a plurality of first adhesive pads, and the plurality of second fastening pads may comprise a plurality of second adhesive pads. In yet other embodiments, the plurality of first fastening pads may include a plurality of first magnetic pads, and the plurality of second fastening pads may comprise a plurality of second magnetic pads.

For example, the base layer may have a rectangle shape, and the plurality of first fastening pads may be respectively carried at corners of the rectangle-shaped base layer. The ostomy bag assembly may comprise a locking ring carried adjacent the second ring sealing feature and configured to be coupled to a belting system.

Also, the first and second ring sealing features may define a tongue and groove interface. The plurality of first fastening pads and the plurality of second fastening pads may be positioned in a diamond arrangement about the stoma. Each fastening pad of the plurality of first fastening pads and the plurality of second fastening pads may be oval-shaped, for example.

Another aspect is directed to a method of making an ostomy appliance. The method may include providing a wafer base plate to be coupled to a stoma of a patient and comprising a base layer having an opening therein positioned inwardly adjacent edges of the base layer, and a first ring sealing feature carried by the base layer and surrounding the opening. The method may include mounting a plurality of first fastening pads to be carried by the base layer surrounding the first sealing feature. The method may include providing an ostomy bag assembly to be coupled to the wafer base plate and comprising a bag having an opening to receive waste from the stoma of the patient, the opening being positioned inwardly from adjacent edges of the bag, and a second ring sealing feature to be coupled to the first ring sealing feature and surrounding the opening of the bag. The method may include mounting a plurality of second fastening pads to be carried by the bag surrounding the second ring sealing feature. The plurality of second fastening pads may be configured to couple to the plurality of first fastening pads so that corresponding portions of the bag are retained against adjacent portions of the wafer base plate as the bag fills with the waste.

### BRIEF DESCRIPTION OF THE DRAWINGS

FIGS. 1-2 are schematic top plan views of an ostomy appliance, according to the prior art.

FIGS. 3-4 are schematic perspective views of an ostomy appliance, according to the prior art.

FIG. 5 is a schematic side view of an ostomy appliance, according to the prior art.

FIGS. 6-7 are schematic top plan views of an ostomy appliance, according to the present disclosure.

FIG. 8 is a schematic perspective view of the ostomy appliance of FIGS. 6-7.

FIGS. 9 and 10 are, respectively, a schematic perspective view and a schematic side view of another embodiment of the ostomy appliance, according to the present disclosure.

FIG. 11 is a schematic perspective view of yet another embodiment of the ostomy appliance, according to the present disclosure.

FIG. 12 is a schematic enlarged view of the ostomy appliance of FIG. 11.

FIG. 13 is a schematic perspective view of yet another embodiment of the ostomy appliance, according to the present disclosure.

### DETAILED DESCRIPTION

The present disclosure will now be described more fully hereinafter with reference to the accompanying drawings, in which several embodiments of the invention are shown. This present disclosure may, however, be embodied in many different forms and should not be construed as limited to the embodiments set forth herein. Rather, these embodiments are provided so that this disclosure will be thorough and complete, and will fully convey the scope of the present disclosure to those skilled in the art. Like numbers refer to like elements throughout, and prime notation is used to indicate similar elements in alternative embodiments.

A desire may exist to address the above issues with a simple approach incorporated as a feature of the two-part appliance system without a requirement to purchase and manage additional products or devices wherein use of such a feature is at the option of the user based on personal preferences. The invention is a simple, secure attachment system which reduces the profile between the two components comprising a pouch and wafer ostomy appliance and provides additional support to the pouch portion of the appliance. The benefits are at least fourfold: (1) minimize the apparent presence of the two-part ostomy appliance to others; (2) lessen or eliminate required changes in choice or fit of clothing; (3) enhance attachment of the pouch to the wafer improving the security of the attachment of the pouch; and (4) smoothing the profile of the pouch, eliminating folds or puckers and allowing waste to flow more freely and smoothly into the pouch.

Additionally, embodiments enhance the support of a pouch portion of the ostomy appliance, thereby improving its function by facilitating the flow of waste into the pouch and at the same time reducing the chance of leakage or separation of the pouch from the wafer portion of the appliance. This invention may address ostomate-expressed concerns related to appliance modification, requirement to purchase additional products, inclusion as an appliance feature, additional cost, utilization of the feature is optional to the user, and use or non-use of the feature will not alter or interfere with appliance functionality.

Embodiments of the components include a Velcro, or another similar Hook and Loop attachment system or another connection element. The Velcro system includes a combination of two separate and distinct components known as Hook and Loop. Pressing the hook portion to the loop portion creates a reliable, secure and removable attachment. Other comparable two-part attachment systems could be utilized other than Velcro brand materials.

Any type of a connection element may be used. Thus, the embodiments disclosed are not limited to a Hook and Loop system. Other non-limiting examples may include a magnetic connection system, an adhesive connection system, a mechanical connection system, etc. Basics of the embodiments comprise an oval shape or semi-circular shaped patches of Hook and Loop materials referred to for convenience in this document as having a “jelly bean” shape. Larger curved shapes may also be used or desirable. An approximate size, depending on appliance location, may vary between 10 mm and 60 mm.

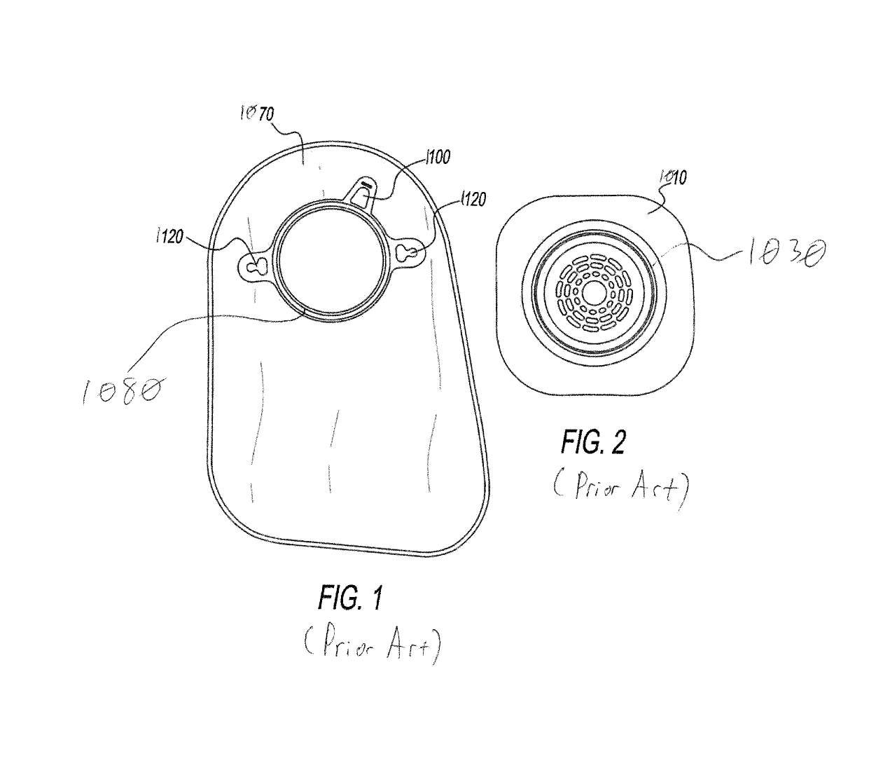

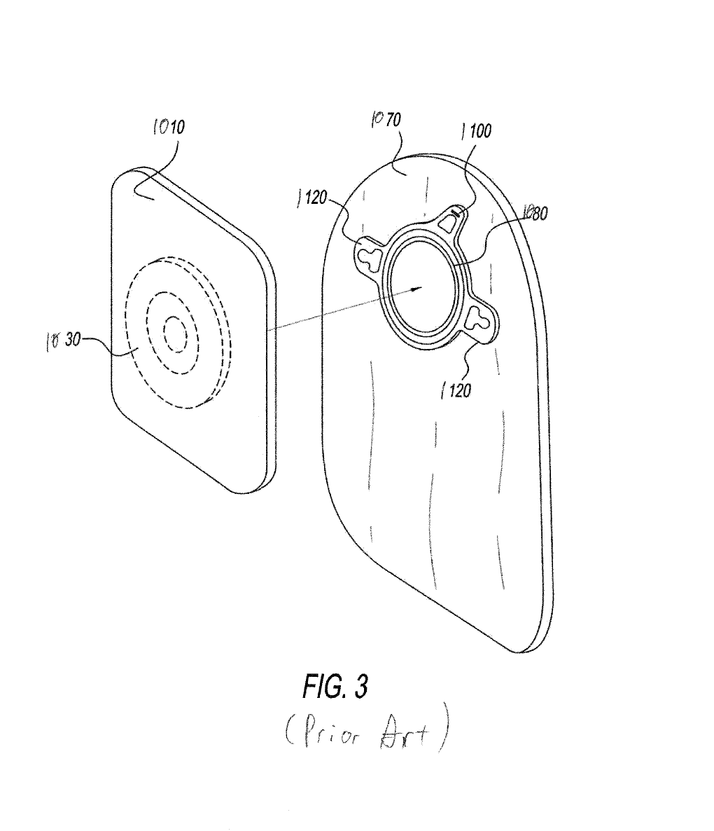

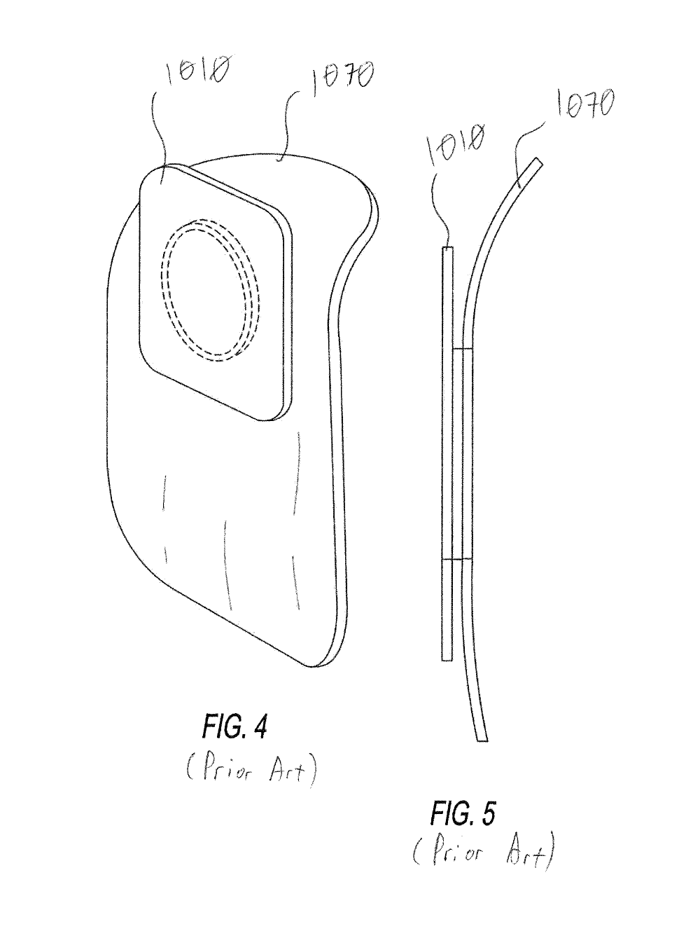

Referring initially to FIGS. 1-5, a typical wafer 1010 and pouch 1070 system is now described. The pouch 1070 illustratively includes a locking ring having tabs 1100, 1120 surrounding a seal 1080. The wafer 1010 includes a seal 1030.

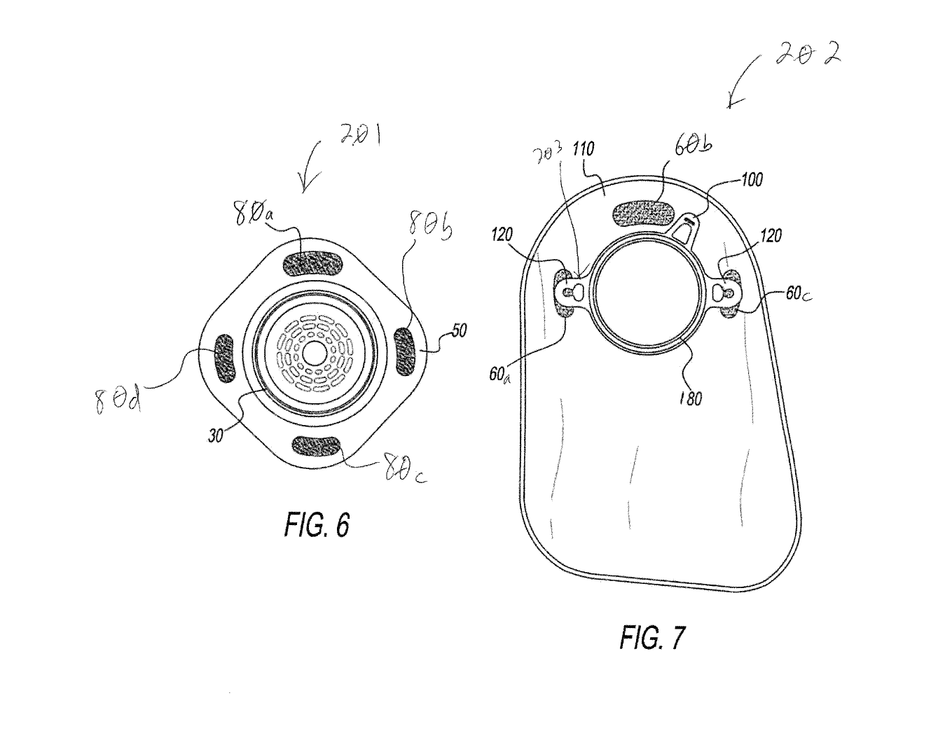

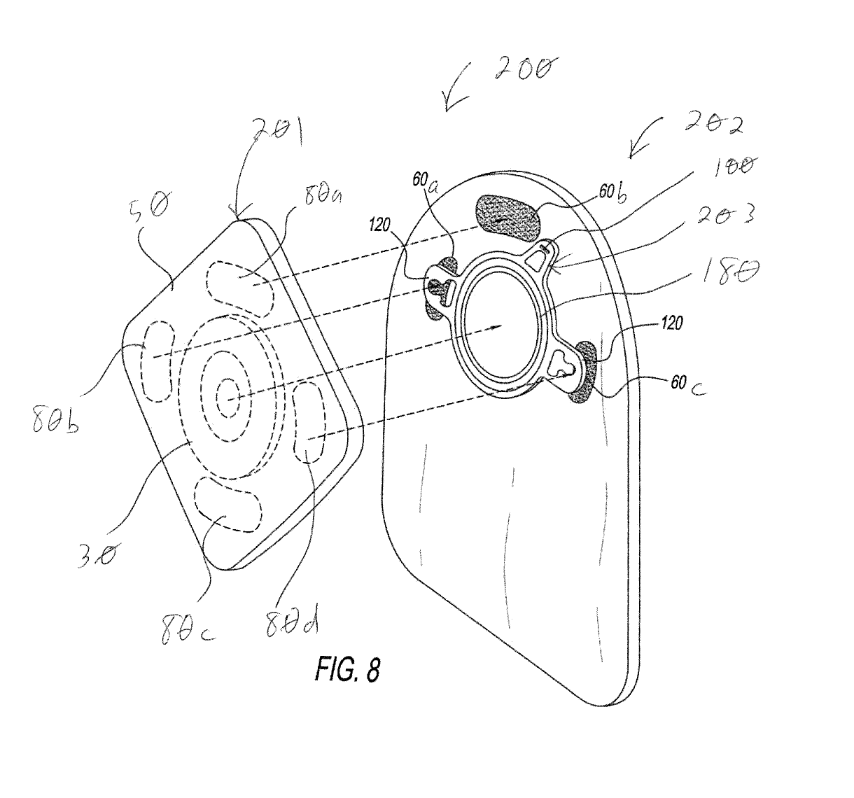

Referring now to FIGS. 6-8, another approach is now described. Location and shape insure maximum closure and holding reliability. Thus, embodiments may include “jelly bean” shaped Hook and Loop patches applied to the wafer and pouch components of the two-part ostomy appliance. Four “jelly bean” shaped Hook and Loop patches (hook portion) may be affixed to the wafer at the four corner locations. Each loop portion may have a pre-installed removable cover to ensure pristine connections when applied to the embodiments disclosed herein. Three “jelly bean” shaped loop patches may be affixed to the pouch. There is no cover on the loop portion. To create a broader horizontal surface and minimize potential leakage, ostomy wound nurses recommend orienting the square wafer to align the diagonal axes vertically and horizontally to create a “diamond” configuration.

With respect to a mechanical wafer attachment, which may have a press on/click configuration, one embodiment of attachment may be to utilize the pressure-sensitive adhesive provided on the Hook and Loop materials to affix them to the wafer. The Hook and Loop patches may be affixed to the wafer at the four corner locations. One embodiment of attachment may be to apply adhesive in a solvent-activated application process to the hook portion of the Hook and Loop patches. Solvent choice depends on product requirements and manufacturing processes.

One embodiment of attachment would be to sew each of the four “jelly bean” shaped Hook and Loop patches to the textile or fabric at each of the four wafer corners. One embodiment of attachment would be to weld, RF seal or heat activate the “jelly bean” shaped Hook and Loop patches to the four wafer corner locations.

With respect to the adhesive wafer attachment, one embodiment of attachment may be to utilize the pressure-sensitive adhesive included on the Hook and Loop patches to affix them to the wafer at the four corner locations. One embodiment of attachment may be to apply adhesive in a solvent-activated application process to the hook portion of the Hook and Loop patches at the four corner locations. Solvent choice depends on product requirements and manufacturing processes. One embodiment of attachment may be to sew the hook portion of each of the four “jelly bean” shaped Hook and Loop patches to the textile or fabric at each of the four wafer corners. One embodiment of attachment may be to weld, RF seal or heat activate the “jelly bean” shaped Hook and Loop patches to the four wafer corner locations.

With respect to pouch locations and attachment embodiments, the “jelly bean” shaped loop portion halves may be affixed to the pouch in 3 locations: top, right and left sides. Means of embodiment of attachments for pouch would be the same as wafer above. Regardless of how the wafer is rotated to orient the diagonal axes to create a diamond configuration, alignment will always occur between three of the four wafer hook patches and the three pouch loop locations: top, right side and left side. Based on personal preference, the ostomate has the choice of attachment at up to three pouch locations. If the ostomate utilizes a belting system, the belt attachment tabs remain unchanged. If a belting system is not utilized, the belt attachment tabs can be removed to reveal right side and left side attachment locations. Removal of the tabs does not create any loss of appliance functionality. Choice of specific attachment methodology is a manufacturer decision dependent upon product requirements and manufacturing processes.

As discussed above, a method of connection may be either mechanical and adhesive or a combination of both. The wafer may be affixed around the stoma location with the diagonal axes of the square wafer oriented vertically and horizontally creating a “diamond” configuration (square system). Depending on his/her attachment preferences, the ostomate may select to use up to three pouch loop locations for attachment (i.e. top, right and left sides). The wafer hook coverage component is removed and discarded. The wafer hook portion is now exposed for attachment with the pouch loop portion. If the ostomate does not use a belting system and desires to attach one or both of the pouch side locations, the corresponding belt attachment tabs must be removed to facilitate attachment with the corresponding wafer location.

Current approaches for adhesive systems currently do not provide a pouch belting option. If there is a pouch venting tab it will be unaffected by embodiments disclosed herein. As for a mechanical press/click connection, the pouch locking ring (positive) may be connected to the wafer locking ring (negative) in a press-on/click manner (“Tupperware” style of closure). The pouch and wafer “jelly bean” shaped patches are connected in the desired locations.

With respect to an adhesive pouch connection, the two adhesive portions are joined together. The pouch and wafer “jelly bean” shaped patches are connected in the desired locations. When maintenance is required (i.e., changing a bag, venting), the pouch and wafer Hook and Loop patches are simply pulled apart.

Referring now to FIGS. 6-8, an ostomy appliance 200 illustratively includes a wafer base plate 201 to be coupled to a stoma of a patient. The wafer base plate 201 illustratively includes a base layer 50 having an opening therein positioned inwardly adjacent edges of the base layer, a first ring sealing feature 30 carried by the base layer and surrounding the opening, and a plurality of first fastening pads 80a-80d carried by the base layer surrounding the first sealing feature.

The ostomy appliance 200 illustratively includes an ostomy bag assembly 202 to be coupled to the wafer base plate 201. The ostomy bag assembly 202 illustratively includes a bag 110 having an opening to receive waste from the stoma of the patient, the opening being positioned inwardly from adjacent edges of the bag, a second ring sealing feature 180 to be coupled to the first ring sealing feature 30 and surrounding the opening of the bag, and a plurality of second fastening pads 60a-60c carried by the bag surrounding the second ring sealing feature. The plurality of second fastening pads 60a-60c are configured to couple to the plurality of first fastening pads 80a-80d so that corresponding portions of the bag 110 are retained against adjacent portions of the wafer base plate 201 as the bag fills with the waste.

The plurality of first fastening pads 80a-80d illustratively comprises a plurality of first Velcro pads, and the plurality of second fastening pads 60a-60c illustratively comprises a plurality of second Velcro pads. As used herein, the term Velcro pad comprises any hook and loop interface, regardless of the manufacturer.

For example, in the illustrated embodiment, the base layer 50 has a rectangle shape, and the plurality of first fastening pads 80a-80d is respectively carried at corners of the rectangle-shaped base layer. In other embodiments, the base layer 50 can have other shapes.

Also, the ostomy bag assembly 202 illustratively includes a locking ring 203 carried adjacent the second ring sealing feature 180 and configured to be coupled to a belting system (now shown). The locking ring 203 illustratively includes a plurality of tabs 120, 100 spaced apart on a periphery thereof. The locking ring 203 is readily removed/installed (e.g. via a press fit interface system) for applications with or without the belting system.

Also, the first and second ring sealing features 30, 180 illustratively define a tongue and groove interface. In other embodiments, the first and second ring sealing features 30, 180 may use an alternative interface so long as it provides a solid seal, for example, a water/air tight seal.

The plurality of first fastening pads 80a-80d and the plurality of second fastening pads 60a-60c may be positioned in a diamond arrangement about the stoma. In the illustrated embodiment, each fastening pad of the plurality of first fastening pads 80a-80d and the plurality of second fastening pads 60a-60c may be oval-shaped, for example. In other embodiments, each fastening pad of the plurality of first fastening pads 80a-80d and the plurality of second fastening pads 60a-60c may have a jellybean shape, a rectangle shape, or a circle shape.

Another aspect is directed to a method of making an ostomy appliance 200. The method may include providing a wafer base plate 201 to be coupled to a stoma of a patient. The wafer base plate 201 includes a base layer 50 having an opening therein positioned inwardly adjacent edges of the base layer, and a first ring sealing feature 30 carried by the base layer and surrounding the opening. The method may include mounting a plurality of first fastening pads 80a-80d to be carried by the base layer 50 surrounding the first sealing feature 30. The method may include providing an ostomy bag assembly 202 to be coupled to the wafer base plate 201. The ostomy bag assembly 202 includes a bag 110 having an opening to receive waste from the stoma of the patient, the opening being positioned inwardly from adjacent edges of the bag, and a second ring sealing feature 180 to be coupled to the first ring sealing feature 30 and surrounding the opening of the bag. The method may include mounting a plurality of second fastening pads 60a-60c to be carried by the bag 110 surrounding the second ring sealing feature 180. The plurality of second fastening pads 60a-60c is configured to couple to the plurality of first fastening pads 80a-80d so that corresponding portions of the bag 110 are retained against adjacent portions of the wafer base plate 201 as the bag fills with the waste.

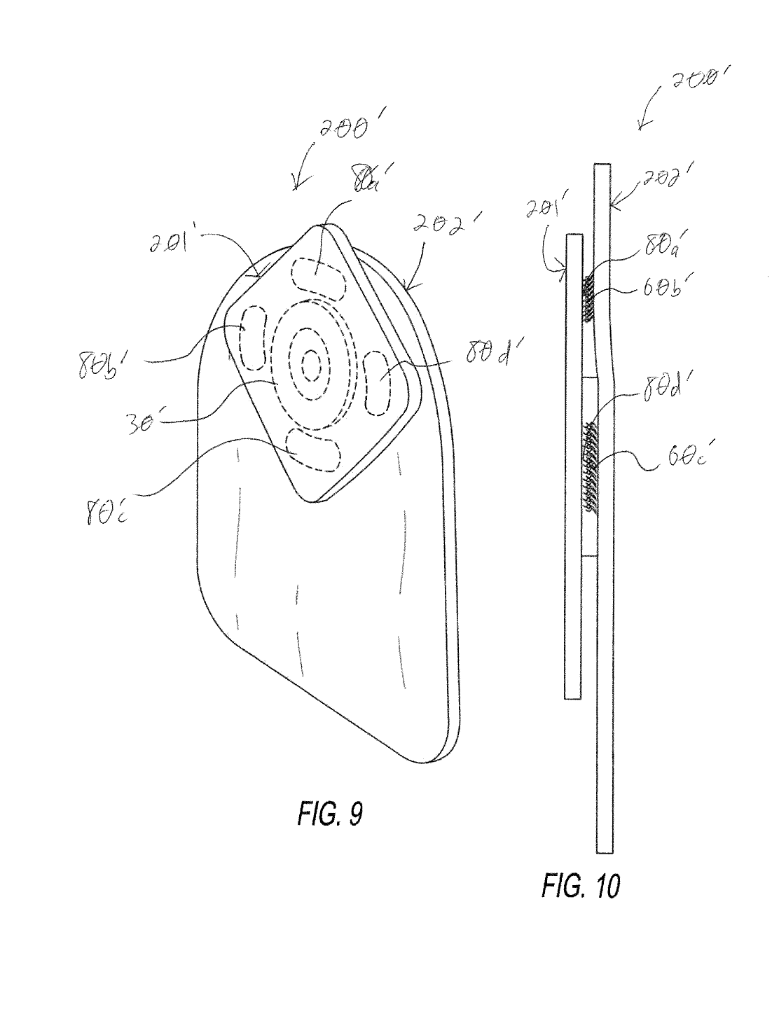

Referring now additionally to FIGS. 9-10, another embodiment of the ostomy appliance 200′ is now described. In this embodiment of the ostomy appliance 200′, those elements already discussed above with respect to FIGS. 6-8 are given prime notation and most require no further discussion herein. This embodiment differs from the previous embodiment in that this ostomy appliance 200′ does not include the locking ring 203 feature.

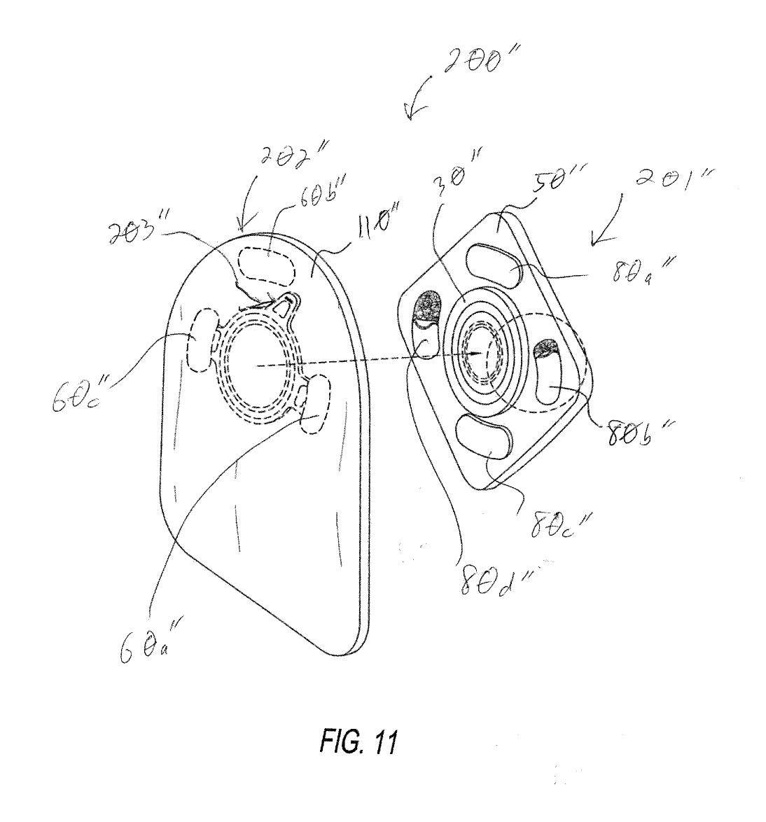

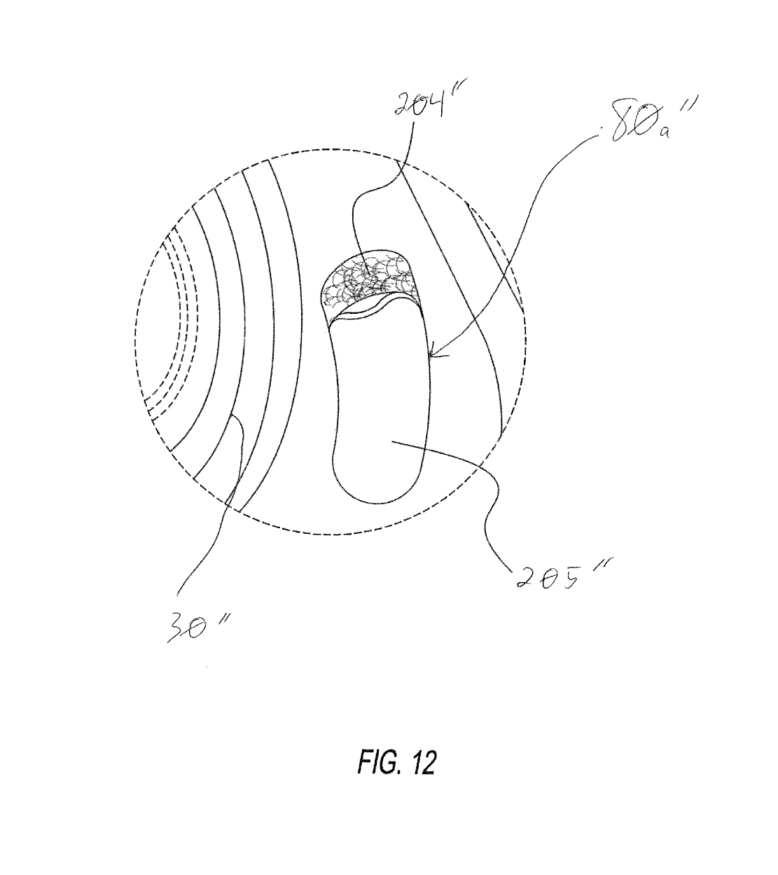

Referring now additionally to FIGS. 11-12, another embodiment of the ostomy appliance 200″ is now described. In this embodiment of the ostomy appliance 200″, those elements already discussed above with respect to FIGS. 6-8 are given double prime notation and most require no further discussion herein. This embodiment differs from the previous embodiment in that the plurality of first fastening pads 80a″-80d″ includes a plurality of first adhesive pads, and the plurality of second fastening pads 60a″-60c″ comprises a plurality of second adhesive pads. As perhaps best seen in FIG. 12, each first fastening pad 80a″-80d″ illustratively includes a release layer 205″, and an adhesive/Velcro pad layer 204″ under the release layer.

In other embodiments, the plurality of first fastening pads 80a″-80d″ includes a plurality of first adhesive pads, and the plurality of second fastening pads 60a″-60c″ comprises a plurality of second adhesive receiving pads (i.e. a surface readily attached to an adhesive contacting surface). In another embodiment, the plurality of first fastening pads 80a″-80d″ includes a plurality of first adhesive receiving pads, and the plurality of second fastening pads 60a″-60c″ comprises a plurality of second adhesive pads.

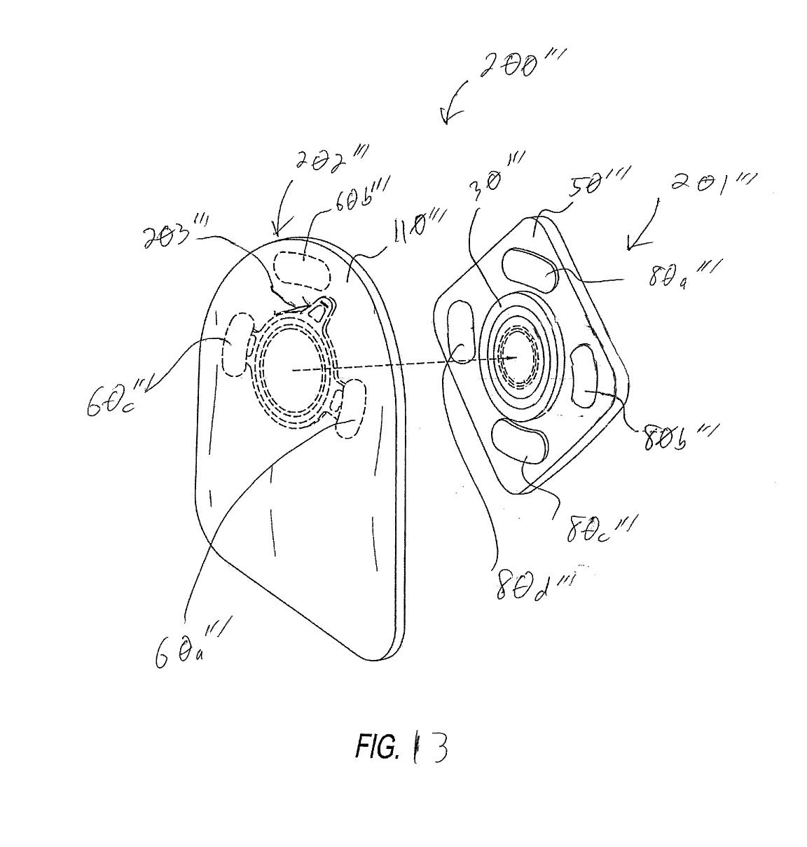

Referring now additionally to FIG. 13, another embodiment of the ostomy appliance 200′″ is now described. In this embodiment of the ostomy appliance 200′″, those elements already discussed above with respect to FIGS. 6-8 are given triple prime notation and most require no further discussion herein. This embodiment differs from the previous embodiment in that the plurality of first fastening pads 80a′″-80d′″ may include a plurality of first magnetic pads, and the plurality of second fastening pads 60a′″-60c′″ may comprise a plurality of second magnetic pads.

Many modifications and other embodiments of the present disclosure will come to the mind of one skilled in the art having the benefit of the teachings presented in the foregoing descriptions and the associated drawings. Therefore, it is understood that the present disclosure is not to be limited to the specific embodiments disclosed, and that modifications and embodiments are intended to be included within the scope of the appended claims.

That which is claimed is:

1. An ostomy appliance comprising:
a wafer base plate to be coupled to a stoma of a patient and comprising
a base layer having an opening therein positioned inwardly adjacent edges of said base layer,
a first ring sealing feature carried by said base layer and surrounding the opening, and
a plurality of first fastening pads carried by said base layer surrounding said first sealing feature; and
an ostomy bag assembly to be coupled to said wafer base plate and comprising
a bag having an opening to receive waste from the stoma of the patient, the opening being positioned inwardly from adjacent edges of said bag,
a second ring sealing feature to be coupled to said first ring sealing feature and surrounding the opening of said bag, and
a plurality of second fastening pads carried by said bag surrounding said second ring sealing feature and configured to couple to said plurality of first fastening pads so that corresponding portions of said bag are retained against adjacent portions of said wafer base plate as the bag fills with the waste.

2. The ostomy appliance of claim 1 wherein said plurality of first fastening pads comprises a plurality of first Velcro pads; and wherein said plurality of second fastening pads comprises a plurality of second Velcro pads.

3. The ostomy appliance of claim 1 wherein said plurality of first fastening pads comprises a plurality of first adhesive pads; and wherein said plurality of second fastening pads comprises a plurality of second adhesive pads.

4. The ostomy appliance of claim 1 wherein said plurality of first fastening pads comprises a plurality of first magnetic pads; and wherein said plurality of second fastening pads comprises a plurality of second magnetic pads.

5. The ostomy appliance of claim 1 wherein said base layer has a rectangle shape; and wherein said plurality of first fastening pads are respectively carried at corners of said rectangle-shaped base layer.

6. The ostomy appliance of claim 1 wherein said ostomy bag assembly comprises a locking ring carried adjacent said second ring sealing feature and configured to be coupled to a belting system.

7. The ostomy appliance of claim 1 wherein said first and second ring sealing features define a tongue and groove interface.

8. The ostomy appliance of claim 1 wherein said plurality of first fastening pads and said plurality of second fastening pads are positioned in a diamond arrangement about the stoma.

9. The ostomy appliance of claim 1 wherein each fastening pad of said plurality of first fastening pads and said plurality of second fastening pads is oval-shaped.

10. An ostomy appliance comprising:
a wafer base plate to be coupled to a stoma of a patient and comprising
a base layer having an opening therein positioned inwardly adjacent edges of said base layer,
a first ring sealing feature carried by said base layer and surrounding the opening, and
a plurality of first oval-shaped Velcro pads carried by said base layer surrounding said first sealing feature; and
an ostomy bag assembly to be coupled to said wafer base plate and comprising
a bag having an opening to receive waste from the stoma of the patient, the opening being positioned inwardly from adjacent edges of said bag,
a second ring sealing feature to be coupled to said first ring sealing feature and surrounding the opening of said bag, and
a plurality of second oval-shaped Velcro pads carried by said bag surrounding said second ring sealing feature and configured to couple to said plurality of first oval-shaped Velcro pads so that corresponding portions of said bag are retained against adjacent portions of said wafer base plate as the bag fills with the waste.

11. The ostomy appliance of claim 10 wherein said base layer has a rectangle shape; and wherein said plurality of first oval-shaped Velcro pads are respectively carried at corners of said rectangle-shaped base layer.

12. The ostomy appliance of claim 10 wherein said ostomy bag assembly comprises a locking ring carried adjacent said second ring sealing feature and configured to be coupled to a belting system.

13. The ostomy appliance of claim 10 wherein said first and second ring sealing features define a tongue and groove interface.

14. The ostomy appliance of claim 10 wherein said plurality of first oval-shaped Velcro pads and said plurality of second oval-shaped Velcro pads are positioned in a diamond arrangement about the stoma.

15. A method of making an ostomy appliance, the method comprising:
providing a wafer base plate to be coupled to a stoma of a patient and comprising
a base layer having an opening therein positioned inwardly adjacent edges of the base layer, and
a first ring sealing feature carried by the base layer and surrounding the opening;
mounting a plurality of first fastening pads to be carried by the base layer surrounding the first sealing feature;
providing an ostomy bag assembly to be coupled to the wafer base plate and comprising
a bag having an opening to receive waste from the stoma of the patient, the opening being positioned inwardly from adjacent edges of the bag, and
a second ring sealing feature to be coupled to the first ring sealing feature and surrounding the opening of the bag; and
mounting a plurality of second fastening pads to be carried by the bag surrounding the second ring sealing feature and configured to couple to the plurality of first fastening pads so that corresponding portions of the bag are retained against adjacent portions of the wafer base plate as the bag fills with the waste.

16. The method of claim 15 wherein the plurality of first fastening pads comprises a plurality of first Velcro pads; and wherein the plurality of second fastening pads comprises a plurality of second Velcro pads.

17. The method of claim 15 wherein the plurality of first fastening pads comprises a plurality of first adhesive pads; and wherein the plurality of second fastening pads comprises a plurality of second adhesive pads.

18. The method of claim 15 wherein the plurality of first fastening pads comprises a plurality of first magnetic pads; and wherein the plurality of second fastening pads comprises a plurality of second magnetic pads.

19. The method of claim 15 wherein the base layer has a rectangle shape; and wherein the plurality of first fastening pads are respectively carried at corners of the rectangle-shaped base layer.

20. The method of claim 15 wherein the ostomy bag assembly comprises a locking ring carried adjacent the second ring sealing feature and configured to be coupled to a belting system.
